(19) United States
(12) Patent Application Publication
(10) Pub. No.: US 2021/0347881 A1
(43) Pub. Date: Nov. 11, 2021

(54) INHIBITION OF KIDNEY DISEASE RELAPSE BY TARGETED CYTOKINE DEPLETION

(71) Applicant: RUSH UNIVERSITY MEDICAL CENTER, Chicago, IL (US)

(72) Inventor: Sumant Singh CHUGH, Oak Park, IL (US)

(21) Appl. No.: 17/262,625

(22) PCT Filed: Jul. 22, 2019
(86) PCT No.: PCT/US2019/042748

§ 371 Date: Jan. 22, 2021

Related U.S. Application Data
(60) Provisional application No. 62/702,975, filed on Jul. 25, 2018.

(51) Int. Cl. C07K 16/24; C07K 16/28; A61P 13/02; A61K 38/17; A61K 45/06

(52) U.S. Cl. CPC C07K 16/246; C07K 16/248; C07K 16/241; C07K 16/2866; A61K 2039/545; A61P 13/02; A61K 38/1793; A61K 45/06; C07K 16/2821

(57) ABSTRACT

Described herein are methods for inhibiting the acute relapse or worsening of glomerular disease in response to viral infections associated with the common cold or flu comprising administration of anti-cytokine antibodies, anti-receptor antibodies, soluble receptors, or blocking agents to a subject in need thereof. Also described are methods for depleting a subjects systemic levels of one or more of cytokines IL-2, IL-6, IL-10, INF-γ, or TNFα, or inhibiting the receptors IL-4R or ICAM-1 comprising contacting the cytokines or receptors with one or more anti-cytokine antibodies, anti-receptor antibodies, soluble receptors, or blocking agents.

### CROSS-REFERENCE TO RELATED APPLICATIONS

This application claims priority to U.S. Provisional Patent Application No. 62/702,975, filed on Jul. 25, 2018, the contents of which are incorporated by reference herein in their entirety.

### GOVERNMENT INTEREST

This invention was made with United States government support under National Institutes of Health/National Institute of Diabetes and Digestive and Kidney Diseases grant numbers R01 DK109713; R01 DK111102; R01 DK101637. The United States government has certain rights in the invention.

### TECHNICAL FIELD

Described herein are methods for inhibiting the acute relapse or worsening of glomerular disease in response to viral infections associated with the common cold or flu comprising administration of anti-cytokine antibodies, anti-receptor antibodies, soluble receptors, or blocking agents to a subject in need thereof. Also described are methods for depleting a subject's systemic levels of one or more of cytokines IL-2, IL-6, IL-10, INF-γ, or TNFα or inhibiting the receptors IL-4R or ICAM-1 comprising contacting the cytokines or receptors with one or more anti-cytokine antibodies, anti-receptor antibodies, soluble receptors, or blocking agents.

### BACKGROUND

Patients with primary glomerular diseases, such as minimal change disease (MCD) and focal and segmental glomerulosclerosis (FSGS), often present with nephrotic syndrome, a constellation of large amounts of protein in the urine (proteinuria), hypoalbuminemia, hyperlipidemia (hypercholesterolemia and hypertriglyceridemia), lipiduria, and edema.

Nephrotic syndrome involves pathology of kidney cells, such as podocytes. Podocytes or visceral epithelial cells are cells in the outer layer of the glomerular capillary loop in the kidneys. As the first step in forming urine, the glomerulus filters large molecules such as proteins from the blood and permits small molecules such as water, salts, and sugars to pass through. Long projections, or “foot processes,” of podocytes wrap around capillaries and rest on the glomerular basement membrane. The foot processes are connected by a porous structure called the slit diaphragm. The innermost layer of the glomerular capillary loop is made of fenestrated endothelial cells that permit passage of small molecules. Kidneys affected by nephrotic syndrome have abnormalities in the glomerular capillary loop that cause leakage of blood proteins into the urine, resulting in proteinuria.

Proteinuria is defined as the presence of an excess of serum proteins in the urine. Albuminuria, a specific type of proteinuria, is a pathological condition wherein albumin is present in the urine. When protein passes into the urine, its plasma concentration decreases and causes water to move into other areas of the body leading to edema. Edema can occur in any body area and is often in response to gravity; it is commonly observed in the feet and legs, in the abdomen (ascites), and around the eyes. Subjects suffering from edema often gain weight, experience fatigue, and may urinate less frequently.

Patients with glomerular diseases often have periods of remission where the disease state is quiescent and symptoms abate or are minimized. When faced with a stressor, such as a viral infection associated with the common cold or flu, the subject often has an acute relapse or worsening of glomerular disease including increased proteinuria, hypoalbuminemia, hyperlipidemia, lipiduria, and edema. The cytokines IL-2, IL-6, IL-10, INF-γ, or TNFα, are increased in periods of viral infection. The acute relapse or worsening of glomerular disease is triggered by the cytokine storm produced in response to the viral infection. Presently there are no known treatments effective for acute relapse or worsening of glomerular disease associated with viral infections.

There is a need for a method to inhibit the relapse or worsening of acute glomerular disease worsening that is initiated by viral infections associated with common cold or flu. In addition, there is a need for a method to deplete IL-2, IL-6, IL-10, INF-γ, or TNFα from systemic circulation or inhibit the receptors IL-4R or ICAM-1 following a viral infection.

### SUMMARY

One embodiment described herein is a method for inhibiting acute relapse or worsening of glomerular disease initiated by viral infection comprising administration of one or more anti-cytokine antibodies, anti-receptor antibodies, soluble receptors, or blocking agents to a subject in need thereof. In one aspect, the anti-cytokine antibodies, anti-receptor antibodies, soluble receptors, or blocking agents comprise one or more of anti-IL-2, anti-IL-6, anti-IL-10, anti-INF-γ, anti-TNFα, soluble TNF receptors, anti-IL-4R, anti-IL-4Ra, anti-IL-6 receptor, anti-ICAM-1, or combinations thereof. In another aspect, the anti-cytokine antibodies, anti-receptor antibodies, soluble receptors, or blocking agents comprise one or more of anti-IL-4R, anti-IL-6, anti-IL-6 receptor, anti-TNFα, soluble TNF receptors, or combinations thereof. In another aspect, the anti-cytokine antibodies, anti-receptor antibodies, soluble receptors, or blocking agents comprise one or more of infliximab (REMICADE®), adalimumab (HUMIRA®), certolizumab pegol (CIMZIA®), etanercept (ENBREL®), siltuximab)(SYLVANT®, tocilizumab (ACTEMRA®), dupilumab (DUPIXENT®), afelimomab, bersanlimab, blazakizumab, fontolizumab, golimumab, nerelimomab, olokizumab, ozoralizumab, pascolizumab, placulumab, sapelizumab, sarilumab, vobarilizumab, combinations thereof, derivatives thereof, or analogues thereof. In another aspect, the glomerular disease comprises nephrotic syndrome. In another aspect, the glomerular disease comprises minimal change disease, focal segmented glomerulosclerosis, membranous nephropathy, membranoproliferative glomerulonephritis, diabetic nephropathy, or lupus nephritis. In another aspect, the glomerular disease comprises acute relapse or worsening of chronic kidney disease caused by minimal change disease, focal segmented glomerulosclerosis, membranous nephropathy, membranoproliferative glomerulonephritis, diabetic nephropathy, or lupus nephritis. In another aspect, the subject is in complete or partial remission of glomerular disease prior to viral infection. In another aspect, the viral infection comprises viruses that induce common cold or flu. In another aspect, the viral infection comprises rhinovirus, human coronavirus, adenovirus, respiratory syncytial virus, enteroviruses other than rhinoviruses, parainfluenza virus, metapneumovirus, influenza, or combination thereof. In another aspect, administration occurs within about 1 hour to about 7 days of onset of the viral infection. In another aspect, administration occurs no later than about 7 days after of onset of the viral infection. In another aspect, the administration comprises parenteral administration. In another aspect, the administration comprises intravenous, intraarterial, intramuscular, intradermal, subcutaneous, or intraperitoneal administration. In another aspect, the administration comprises intravenous administration. In another aspect, the subject is a human.

Another embodiment described herein is a method for depleting a subject's systemic levels of one or more of cytokines IL-2, IL-6, IL-10, INF-γ, or TNFα, or inhibiting the receptors IL-4R or ICAM-1 comprising contacting the cytokines or receptors with one or more anti-cytokine antibodies, anti-receptor antibodies, soluble receptors, or blocking agents. In one aspect, the anti-cytokine antibodies, anti-receptor antibodies, soluble receptors, or blocking agents comprise anti-IL-2, anti-IL-6, anti-IL-10, anti-INF-γ, anti-TNFα, anti-IL-4R, anti-ICAM-1, or combinations thereof. In another aspect, the contacting comprises parenteral administration of one or more of anti-cytokine antibodies, anti-receptor antibodies, soluble receptors, or blocking agents. In another aspect, the parenteral administration comprises intravenous, intraarterial, intramuscular, intradermal, subcutaneous, or intraperitoneal administration. In another aspect, the parenteral administration comprises intravenous administration. In another aspect, the subject is experiencing acute relapse onset or worsening of glomerular disease. In another aspect, the subject is in complete or partial remission of glomerular disease prior to worsening of glomerular disease. In another aspect, the subject is a human.

Another embodiment described herein is a method for blocking intracellular effects of IL-2, IL-6, IL-10, INF-γ, TNFα, IL-4R, or ICAM-1 in kidney disease by contacting one or more IL-2, IL-6, IL-10, INF-γ, TNFα, IL-4R, or ICAM-1 receptors with one or more anti-cytokine antibodies, anti-receptor antibodies, soluble receptors, or blocking agents. In one aspect, the blocking agent comprises a decoy receptor for IL-2, IL-6, IL-10, INF-γ, TNFα, IL-4R, or ICAM-1. In another aspect, the blocking agent comprises one or more small molecules.

Another embodiment described herein is a composition comprising a mixture of one or more anti-cytokine antibodies, anti-receptor antibodies, soluble receptors, or blocking agents comprising anti-IL-2, anti-IL-6, anti-IL-10, anti-INF-γ, anti-TNF, anti-IL-4R, or anti-ICAM-1; and optionally, one or more pharmaceutically acceptable excipients. In one aspect, the composition comprises 1 mg to 10,000 mg of each antibody, soluble receptor, or blocking agent. In another aspect, the composition dose is about 0.01 mg/kg to about 200 mg/kg. In another aspect, the composition dose is about 0.5 mg/kg to about 50 mg/kg. In another aspect, the composition is a liquid or a lyophylized concentrate.

Another embodiment described herein is a kit for depleting a subject's systemic levels of one or more of cytokines IL-2, IL-6, IL-10, INF-γ, or TNFα or inhibiting receptors IL-4R or ICAM-1 comprising one or more receptacles comprising a mixture of one or more anti-cytokine antibodies, anti-receptor antibodies, soluble receptors, or blocking agents comprising anti-IL-2, anti-IL-6, anti-IL-10, anti-INF-γ, anti-TNF, anti-IL-4R, or anti-ICAM-1, blocking agents, or a combination thereof, and optionally, a delivery device, a diluent, instructions for use, or a label.

### BRIEF DESCRIPTION OF THE DRAWINGS

FIG. 1A shows albuminuria levels after intravenous injection of a panel of circulating cytokines and receptors (dose “X”) or control saline in Balb/c and Balb/cJ mice (n=5 mice per group; (*p<0.05).

FIG. 1B shows albuminuria levels after injecting each individual component of dose “X” (e.g., IL-2, IL-4R, IL-6, IL-10, INF-γ, TNFα, ICAM-1) into Balb/cJ mice (n=4 mice per group).

FIG. 2A shows albuminuria levels after injecting declining percentages (100, 50, 10 and 1) of dose “X” into Balb/cJ mice (n=3 mice per group; *p<0.05, ***p<0.001).

FIG. 2B shows albuminuria levels after eliminating individual components (e.g., IL-4R, IL-6, TNFα) from the cytokine cocktail dose X/2 injected into Balb/c and Balb/cJ mice (n=5 mice per group; *p<0.05).

FIG. 3A shows that the injection of Balb/cJ mice with a small dose of an anti-TNFα antibody (25 μg) or control IgG one hour after injection of cytokine cocktail dose (X/2) resulted in significantly lower Day 1 albuminuria in the anti-TNFα group (n=7 mice per group; *p<0.05).

FIG. 3B shows albuminuria levels after injecting the cytokine cocktail dose X/15 into podocyte specific Zhx2 deficient and foxed control mice (*p<0.05 on Day 0 for both groups, #p<0.05 between Day 0 and Day 1 for Zhx2 flox/flox/Cre).

FIG. 4A shows albuminuria levels after injecting mice deficient in Enpep, Zhx2 or both with the cytokine cocktail dose X/5 (n=7 mice per group). The right sub-panel in FIG. 4A demonstrates greater than 5-fold higher mean albuminuria at a dose of X/2 (n=6 mice per group) compared to a dose of X/5 (*p<0.05; **p<0.01).

FIG. 4B shows the fold-change of albuminuria levels after injecting mice deficient in Enpep, Zhx2 or both with the cytokine cocktail dose X/5 (n=7 mice per group; *p<0.05; **p<0.01).

FIG. 5 shows the distribution of podocyte Zhx proteins (Zhx1 and Zhx3) in the peripheral and nuclear compartments 24 hours after injecting the cytokine cocktail in Balb/c and Balb/cJ mice. FIG. 5A shows the distribution of Zhx1 and FIG. 5B shows the distribution of Zhx3. White arrows in FIG. 5A show increased nuclear Zhx1 in podocyte nuclei of Balb/cJ mice treated with the cytokine cocktail. Scale bars: 12.5 μm for “Saline” and left “Cytokine” columns in FIG. 5A and FIG. 5B; 7.9 μm for the FIG. 5A right Cytokine column.

FIG. 6 shows the peak change in proteinuria from baseline in Buffalo/Mna rats up to 7 days after injection of the rat cytokine cocktail minimum nephritogenic dose X/50 (n=7 rats; *p<0.05).

FIG. 7 shows a schematic representation of the cytokine storm after a common cold, and rationale for the cytokine cocktail. Rhinoviruses are the most common cause of a common cold. Rhinoviruses types A and B bind to their receptor Intercellular Adhesion Molecule 1 (ICAM-1) in the nasal epithelium to enter the cells. TNFα, IL-6, and IL-10 are secreted as part of innate immunity, whereas IL-2 and IFN-γ are secreted as part of adaptive immunity. IL-4R is increased in all forms of rhinitis. ICAM-1 is released from injured or infected nasal epithelium. Collectively, these cytokines and soluble receptors form a complex that interacts with the podocyte surface to induce relapse or worsening of podocyte diseases. Therapeutic intervention using antibodies against any member of the cytokine cocktail as shown herein prevents or reduces the intensity of relapse or disease worsening.

### DETAILED DESCRIPTION

One embodiment described herein is a method for inhibiting the acute relapse or worsening of glomerular disease comprising administration of one or more anti-cytokine antibodies, anti-receptor antibodies, soluble receptors, or blocking agents to a subject in need thereof. In one aspect, the worsening or relapse is initiated by a viral infection. In another aspect, the viral infection is associated with the common cold or flu. For the avoidance of doubt, this method is not intended to treat or cure active glomerular disease or inhibit the progression of active glomerular disease. This method inhibits the acute relapse or worsening of glomerular disease that is exacerbated by the cytokine storm resulting from a viral infection associated with a common cold of flu.

As used herein the terms “cytokine” or “cytokines” refer to the interleukins, interferons, tumor necrosis factor (TNF), growth factors, or other molecules that can exert an effect on other cells.

As used herein the term “receptor” refers to receptors of cytokines or other molecules, such as interleukin 4 receptor (including IL-4Ra), intracellular adhesion molecules (ICAMs), or other cell surface receptors.

As used herein, the term “soluble receptor” refers to a receptor, typically recombinant, that can interact with a cytokine. Examples include soluble tumor necrosis factor receptor (TNF receptor) or IL-4R. In addition, the term soluble receptor also refers to an alternative to an anti-cytokine antibody.

As used herein the terms “anti-cytokine antibodies,” or “anti-cytokine antibody” refer to antibodies that recognize one or more cytokines such as interleukins, interferons, tumor necrosis factor (TNF), growth factors, or other molecules that can exert an effect on other cells. The antibodies may be natural, polyclonal, monoclonal, single chain, recombinant, or humanized. Humanized antibodies are particularly useful.

As used herein the terms “anti-receptor antibodies,” or “anti-receptor antibody” refer to antibodies that recognize one or more cytokine receptor molecules such interleukin 4 receptors (anti-IL-4R), intracellular adhesion molecules (ICAM-1), or other cytokine or cell surface receptors.

As used herein the term “blocking agent” refers to a molecule that can interact with a cytokine, cytokine receptor, soluble receptor, cell-surface receptor, or the like and inhibit its natural function. Typically, inhibition involves blocking the ability of the molecule to bind its receptor or natural ligand. Blocking agents may be biomolecules such as proteins or small molecules.

As used herein the terms “acute relapse” or “relapse” refer to the deterioration in health and an intensification of disease state or symptoms after a period of improvement or remission. “Acute relapse” refers specifically to the sudden worsening or intensification of a disease state or symptoms, often in response to a stressor. In one aspect, the stressor is a viral infection. Additional stressors comprise other infections, inflammation, diabetes, autoimmune disorders, or trauma.

As used herein, the term “worsening,” specifically, “worsening of glomerular disease” refers to the further intensification of the disease state or symptoms.

As used herein, the term “remission” refers to the diminution of the seriousness or intensity of a disease or disease symptoms. Remission can be complete or partial where some symptoms diminish but continue persist. In one aspect, remission refers to the absence of symptoms of glomerular disease.

As used herein, the terms “treatment”, “treat,” and “treating” refers a course of action (such as administering a compound or pharmaceutical composition) initiated after the onset of a symptom, aspect, or characteristics of a disease or condition so as to eliminate or reduce such symptom, aspect, or characteristics. Such treating need not be absolute to be useful.

As used herein, the terms “in need thereof” or “in need of treatment” refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease or condition that is treatable by a method or compound of the disclosure.

The terms “subject” or “patient” as used herein refers to any animal, including mammals, such as mice, rats, other rodents, rabbits, dogs, cats, swine, cattle, sheep, horses, non-human primates, or humans. In one aspect, the subject is a human. In another aspect, the subject is a human child or adult. In another aspect, the subject is in need thereof or in need of treatment.

As used herein, the term “therapeutically effective amount” refers to an amount of a compound, either alone or as a part of a pharmaceutical composition, that is capable of having any detectable, positive effect on any symptom, aspect, or characteristics of a disease or condition. Such effect need not be absolute to be beneficial. When referring to an anti-cytokine antibody, anti-receptor antibody, soluble receptor, or blocking agent, the term “therapeutically effective amount” refers to an amount of such species sufficient to stem the onset of acute glomerular disease relapse. The therapeutically effective amount will vary for each anti-cytokine antibody, anti-receptor antibody, soluble receptor, or blocking agent. For example, a composition comprising three specific anti-cytokine antibodies, will potentially have three independent therapeutically effective amounts—one for each anti-cytokine antibody.

The terms “about” and “approximately” as used herein refer an acceptable degree of error or variation for the quantity measured given the nature or precision of the measurements. Typical, exemplary degrees of error or variation are within 20% or within 10% of a given value or range of values. For biological systems, the term “about” refers to an acceptable standard deviation of error, preferably not more than 2-fold of a give value. Numerical quantities given herein are approximate unless stated otherwise, meaning that the term “about” or “approximately” can be inferred when not expressly stated.

Results described herein show that glomerular disease symptoms such as albuminuria can be induced in Balb/cJ mice, which have reduced expression of transcriptional factor ZHX2, by the administration of a cytokine cocktail of IL-2, IL-6, IL-10, INF-γ, TNFα, IL-4R, and ICAM-1. The individual cytokines did not induce albuminuria. Based on these results, a method for inhibiting acute relapse or worsening of glomerular disease initiated by viral infection was developed.

One embodiment described herein is a method for inhibiting acute relapse or worsening of glomerular disease initiated by viral infection comprising administration of one or more anti-cytokine antibodies, anti-receptor antibodies, soluble receptors, or blocking agents to a subject in need thereof. In one aspect, the anti-cytokine antibodies, anti-receptor antibodies, soluble receptors, or blocking agents comprise one or more of anti-IL-2, anti-IL-6, anti-IL-10, anti-INF-γ, anti-TNFα, soluble TNF receptors, anti-IL-4R, anti-IL-6 receptor, anti-ICAM-1, or combinations thereof. In another aspect, the anti-cytokine antibodies, anti-receptor antibodies, soluble receptors, or blocking agents comprise one or more of anti-IL-4R, anti-IL-6, anti-IL-6 receptor, anti-TNFα, soluble TNF receptors, or combinations thereof. In another aspect, the anti-cytokine antibodies, anti-receptor antibodies, soluble receptors, or blocking agents comprise one or more of infliximab (REMICADE®), adalimumab (HUMIRA®), certolizumab pegol (CIMZIA®), etanercept (ENBREL®), siltuximab (SYLVANT®), tocilizumab (ACTEMRA®), dupilumab, (DUPIXENT®), afelimomab, bersanlimab, blazakizumab, fontolizumab, golimumab, nerelimomab, olokizumab, ozoralizumab, pascolizumab, placulumab, sapelizumab, sarilumab, vobarilizumab, combinations thereof, derivatives thereof, or analogues thereof.

In one embodiment, the glomerular disease comprises nephrotic syndrome, which is characterized by proteinuria, hypoalbuminemia, hyperlipidemia (hypercholesterolemia and hypertriglyceridemia), lipiduria, edema, or a combination thereof. In some aspect, the glomerular disease comprises minimal change disease, focal segmented glomerulosclerosis, membranous nephropathy, membranoproliferative glomerulonephritis, diabetic nephropathy, or lupus nephritis. In another aspect, the glomerular disease comprises acute relapse or worsening of chronic kidney disease caused by minimal change disease, focal segmented glomerulosclerosis, membranous nephropathy, membranoproliferative glomerulonephritis, diabetic nephropathy, or lupus nephritis.

In one embodiment, prior to acute relapse or worsening of glomerular disease, the subject is in complete or partial remission from the disease state. In one aspect, the nephrotic syndrome symptoms are abated or are reduced during the remission period. In another aspect, the glomerular disease is in the quiescent state. In another aspect, the glomerular disease is persistant but in an attenuated disease state.

In one embodiment, the acute relapse or worsening of glomerular disease results from viral infection. In one aspect, the viral infection comprises viruses that induce common cold or flu. In another aspect, the viral infection comprises rhinovirus, human coronavirus, adenovirus, respiratory syncytial virus, enteroviruses other than rhinoviruses, parainfluenza virus, metapneumovirus, influenza, or combination thereof.

In one embodiment, the administration of the anti-cytokine antibodies, anti-receptor antibodies, soluble receptors, or blocking agents occurs within about 1 hour to about 7 days of the onset of the viral infection. In one aspect the administration occurs after about 1 h, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, or about 24 h of the onset of viral infection. In another aspect, the administration occurs after about 0.1 day, about 0.25 day, about 0.5 day, about 0.75 day, about 1 day, about 2 days, about 3 days, about 4 days, about 5 days, about 6 days, or about 7 days after the onset of viral infection. In one aspect, the onset of viral infection is identified by the emergence of viral infection symptoms. Viral infection symptoms include but are not limited to runny nose, sneezing, sore throat, scratchy throat, cough, congestion, postnasal drip, watery eyes, muscle aches, joint aches, headache, fever, tiredness, malaise, nausea, stomachache, diarrhea, or vomiting, a combination thereof.

In another embodiment, the administration of the anti-cytokine antibodies, anti-receptor antibodies, soluble receptors, or blocking agents occurs no later than about 7 days after the onset of viral infection. In another aspect, the administration occurs no later than about 1 day, about 2 days, about 3 days, about 4 days, about 5 days, about 6 days, or about 7 days after onset of the viral infections. In another aspect, the administration occurs no later than about 1 week from the onset of the viral invention. In another aspect, administration occurs no later than about 48 hours after the onset of the viral invention. In one aspect, the onset of viral infection is identified by the emergence of viral infection symptoms including include runny nose, sneezing, sore throat, scratchy throat, cough, congestion, postnasal drip, watery eyes, muscle aches, joint aches, headache, fever, tiredness, malaise, nausea, stomachache, diarrhea, or vomiting, a combination thereof.

In another embodiment, the administration of the anti-cytokine antibodies, anti-receptor antibodies, soluble receptors, or blocking agents is by any means effective to administer the medicaments. In one aspect, the administration comprises parenteral administration. In another aspect, the administration comprises intravenous, intraarterial, intramuscular, intradermal, subcutaneous, or intraperitoneal administration. In another aspect, the administration comprises intravenous administration. In another aspect, the administration comprises intravenous infusion.

In another embodiment described herein, the subject is a mouse, rat, other rodent, rabbit, dog, cat, pig, cow, sheep, horse, non-human primate, or human. In one embodiment, the subject is a human or a human in need thereof. In another aspect, the subject is a human child or adult in need thereof. As used herein, the phrase “in need thereof” indicates that the subject is need of treatment as determined by a physician.

Another embodiment described herein is a method for depleting a subject's systemic levels of one or more of cytokines IL-2, IL-6, IL-10, INF-γ, or TNFα, or inhibiting the receptors IL-4R or ICAM-1 comprising contacting the cytokines or receptors with one or more anti-cytokine antibodies, anti-receptor antibodies, soluble receptors, or blocking agents. In one aspect, the anti-cytokine antibodies, anti-receptor antibodies, soluble receptors, or blocking agents comprise one or more of anti-IL-2, anti-IL-6, anti-IL-10, anti-INF-γ, anti-TNFα, soluble TNF receptors, anti-IL-4R, anti-IL-6 receptor, anti-ICAM-1, or combinations thereof. In another aspect, the anti-cytokine antibodies, anti-receptor antibodies, soluble receptors, or blocking agents comprise one or more of anti-IL-4R, anti-IL-6, anti-IL-6 receptor, anti-TNFα, soluble TNF receptors, or combinations thereof. In another aspect, the anti-cytokine antibodies, anti-receptor antibodies, soluble receptors, or blocking agents comprise one or more of infliximab (REMICADE®), adalimumab (HUMIRA®), certolizumab pegol (CIMZIA®), etanercept (ENBREL®), siltuximab (SYLVANT®), tocilizumab (ACTEMRA®), dupilumab (DUPIXENT®), afelimomab, bersanlimab, blazakizumab, fontolizumab, golimumab, nerelimomab, olokizumab, ozoralizumab, pascolizumab, placulumab, sapelizumab, sarilumab, vobarilizumab, combinations thereof, derivatives thereof, or analogues thereof. In another aspect, the contacting comprises parenteral administration of one or more of anti-cytokine antibodies, anti-receptor antibodies, soluble receptors, or blocking agents to the subject which permits the anti-cytokine antibodies, anti-receptor antibodies, soluble receptors, or blocking agents to interact with IL-2, IL-6, IL-10, INF-γ, TNFα, IL-4R, or ICAM-1 and neutralize the cytokine or receptor ligand preventing binding and downstream effects. In one aspect, the parenteral administration comprises intravenous administration. In another aspect, the subject is experiencing acute relapse onset or worsening of glomerular disease. In one aspect, the he subject is experiencing acute relapse onset or worsening of glomerular disease owing to a viral infection associated with the common cold or flu. In another aspect, subject is in complete or partial remission of glomerular disease prior to worsening of glomerular disease. In one aspect, the subject is a human.

Another embodiment described herein is a method for blocking intracellular effects of IL-2, IL-6, IL-10, INF-γ, TNFα, IL-4R, or ICAM-1 in kidney disease by contacting one or more IL-2, IL-6, IL-10, INF-γ, TNFα, IL-4R, or ICAM-1 receptors with one or more blocking agents. In one aspect, the blocking agent comprises anti-cytokine antibodies, soluble receptors, or decoy receptors for IL-2, IL-6, IL-10, INF-γ, TNFα, IL-4R, or ICAM-1. In another aspect, the blocking agent comprises one or more small molecules that are capable of binding to the receptor and blocking interaction with the cytokine or receptor ligand. In one aspect, the blocking agent comprises anti-IL-2, anti-IL-6, anti-IL-10, anti-INF-γ, anti-TNFα, anti-IL-4R, anti-ICAM-1, or combinations thereof.

Another embodiment described herein is a pharmaceutical composition comprising a mixture of one or more anti-cytokine antibodies, anti-receptor antibodies, soluble receptors, or blocking agents comprising anti-IL-2, anti-IL-6, anti-IL-10, anti-INF-γ, anti-TNF, anti-IL-4R, or anti-ICAM-1; and optionally, one or more pharmaceutically acceptable excipients. In one aspect, the composition comprises 1 mg to 10,000 mg of each antibody. In another aspect, the composition dose is about 0.01 mg/kg to about 200 mg/kg. In another aspect, the composition dose is about 5 mg/kg. In another aspect, the composition is a liquid or a lyophylized concentrate.

Another embodiment described herein is a kit for depleting a subject's systemic levels of one or more of cytokines IL-2, IL-6, IL-10, INF-γ, or TNFα or receptors IL-4R or ICAM-1 comprising one or more receptacles comprising a composition comprising anti-cytokine antibodies anti-IL-2, anti-IL-6, anti-IL-10, anti-INF-γ, or anti-TNF antibodies or anti-receptor antibodies for IL-4R or ICAM-1, combinations thereof and optionally, a delivery device, a diluent, instructions for use, or a label.

Pharmaceutical compositions suitable for administration by injection include sterile aqueous solutions, suspensions, or dispersions and sterile powders or lyophilisates for the extemporaneous preparation of sterile injectable solutions or dispersion.

For intravenous administration, suitable carriers include physiological saline, phosphate buffered saline (PBS), Ringer's solution, or water for injection. In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, buffers, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, or by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, amino acids, sorbitol, sodium chloride, or combinations thereof in the composition. Prolonged absorption of the injectable compositions can be brought about by including in the composition an agent that delays absorption, for example, aluminum monostearate and gelatin.

Certain injectable compositions are aqueous isotonic solutions or suspensions. Such compositions may be sterilized and/or contain adjuvants, such as preservatives, stabilizers, wetting agents, emulsifying agents, solution promoters, salts for regulating the osmotic pressure and/or buffers. In addition, they may also contain other therapeutically valuable substances. Said compositions are prepared according to conventional methods, respectively, and contain about 0.1-75%, or contain about 1-50%, of the active ingredient.

Sterile injectable solutions or suspensions can be prepared by incorporating the anti-cytokine antibodies, anti-receptor antibodies, soluble receptors, or blocking agents in the required amount in an appropriate solvent with one or a combination of ingredients, as required, followed by filtration sterilization. Generally, solutions or suspensions are prepared by incorporating the active compound into a sterile vehicle such as sterile PBS and any excipients. In the case of sterile powders for the preparation of sterile injectable solutions, the preferred preparation methods are vacuum drying and freeze-drying which yields a powder of the active ingredient plus any additional desired ingredient from a previously sterile-filtered solution thereof.

The effective amount of a pharmaceutical composition to be employed therapeutically will depend, for example, upon the therapeutic context and objectives. One skilled in the art will appreciate that the appropriate dosage levels for treatment will thus vary depending, in part, upon the therapeutic agent incorporated into the pharmaceutical composition, the indication for which the therapeutic agent is being used, the route of administration, and the size (body weight, body surface, or organ size) and condition (the age and general health) of the patient. Accordingly, the clinician can titer the dosage and modify the route of administration to obtain the optimal therapeutic effect.

Anti-cytokine antibodies, anti-receptor antibodies, soluble receptors, or blocking agents can be produced using standard monoclonal antibody (particularly humanized monoclonal antibodies) techniques, protein engineering, and general molecular biological techniques know to those having ordinary skill in the art.

Several exemplary FDA-approved anti-cytokine antibodies, anti-receptor antibodies, soluble receptors, or blocking agents useful for the methods described herein include infliximab (REMICADE®) (anti-TNFα); adalimumab (HUMIRA®) (anti-TNFα); certolizumab pegol (CIMZIA®) (anti-TNFα); etanercept (ENBREL®) (TNF receptor fusion protein); siltuximab (SYLVANT®) (anti-IL-6); tocilizumab (ACTEMRA®) (anti-IL-6 receptor); or dupilumab (DUPIXENT®) (anti-IL-4Ra). Relevant dose and concentration parameters of these agents are provided in Table 1.

The FDA prescribing information, e.g., labels, for infliximab (REMICADE®), adalimumab (HUMIRA®), certolizumab pegol (CIMZIA®), etanercept (ENBREL®), siltuximab (SYLVANT®), tocilizumab (ACTEMRA®), and dupilumab (DUPIXENT®) are each incorporated herein by express reference thereto for their specific teachings.

TABLE 1
FDA-approved anti-cytokine or anti-receptor
antibodies or soluble receptors

| | | Indicated Dose; | Concentration, |
|---|---|---|---|
| Therapeutic | Target | Total Dose | Vol. |
| Infliximab | TNF | 3-10 mg/kg/0, 2, 6 | 5 mg/mL; 20 mL |
| (REMICADE ®) | | weeks; then every 8 | |
| | | weeks | |
| Adalimumab | TNF | 0.6 mg/kg; | 50 mg/mL; 0.4 or |
| (HUMIRA ®) | | 40 mg/2 weeks | 0.8 mL |
| Certolizumab pegol | TNF | 6 mg/kg; | 200 mg/mL; 1 mL |
| (CIMZIA ®) | | 400 mg/2 weeks | |
| Etanercept | TNF | 0.7 mg/kg; | 50 mg/mL; 0.5 or |
| (ENBREL ®) | | 50 mg/week | 1.0 mL |
| Siltuximab | IL-6R | 11 mg/kg/3 weeks | 20 mg/mL; 5 or 20 |
| (SYLVANT ®) | | | mL |
| Tocilizumab | IL-6R | 4-12 mg/kg/4 weeks | 20 mg/mL or 180 |
| (ACTEMRA ®) | | | mg/mL; 50 or 100 |
| | | | mL |
| Dupilumab | IL-4Rα | 8.6 or 4.2 or mg/kg; | 150 mg/mL; 2 mL |
| (DUPIXENT ®) | | 600 mg initially, | |
| | | 300 mg/2 weeks | |

Italicized doses (mg/kg) were calculated based on the total indicated dose using an average human body mass of 70 kg. Non-italicized doses were provided in the label.

As can be observed from Table 1, the dose of each therapeutic agent varies in its administration dose, recurrent dosing schedule, and concentration. Further, when such protein therapeutic agents are administered to subjects with glomerular disease, higher doses are often required to achieve a therapeutic effect. Without being bound by any theory, this is believed to be due to the leakage of the therapeutic protein(s) (among other blood proteins) into the urine owing to the abnormalities in the glomerular capillary loop that are responsible for causing proteinuria. Accordingly, doses of the anti-cytokine antibodies, anti-receptor antibodies, soluble receptors, or blocking agents higher than indicated in the label can be administered to compensate for the loss of the therapeutic agent via the kidney.

Other anti-cytokine antibodies, anti-receptor antibodies, or soluble receptors are approved for human use throughout the world. For example the following compounds (target) are useful: afelimomab (TNFα); bersanlimab (ICAM-1); clazakizumab (IL-6); fontolizumab (INF-γ); golimumab (TNFα); nerelimomab (TNFα); olokizumab (IL-6); ozoralizumab (TNFα); pascolizumab (IL-4); placulumab (TNF); sapelizumab (IL-6R); sarilumab (IL-6); vobarilizumab (IL-6R), among others. These and other analagous molecules are useful for the methods and compositions described herein.

One embodiment described herein, is a pharmaceutical composition comprising 1 mg to 10,000 mg of each of one or more anti-cytokine antibodies, anti-receptor antibodies, soluble receptors, or blocking agents. In one aspect, the composition comprises about 1 mg, about 2 mg, about 5 mg, about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, about 1000 mg, about 2000 mg, about 3000 mg, about 4000 mg, about 5000 mg, about 6000 mg, about 7000 mg, about 8000 mg, about 9000 mg, about 10000 mg, or even greater amounts for each of the one or more anti-cytokine antibodies, anti-receptor antibodies, soluble receptors, or blocking agents.

Another embodiment described herein, is a pharmaceutical composition wherein the administration comprises one or more anti-cytokine antibodies, anti-receptor antibodies, soluble receptors, or blocking agents comprising about 0.01 mg/kg, about 0.05 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 40 mg/kg, about 50 mg/kg, about 60 mg/kg, about 70 mg/kg, about 80 mg/kg, about 90 mg/kg, about 100 mg/kg, about 120 mg/kg, about 150 mg/kg, about 175 mg/kg, about 200 mg/kg, about 250 mg/kg, about 300 mg/kg, about 400 mg/kg, about 500 mg/kg, or even greater for each of the one or more anti-cytokine antibodies, anti-receptor antibodies, soluble receptors, or blocking agents.

Another embodiment described herein, is a pharmaceutical composition comprising one or more anti-cytokine antibodies, anti-receptor antibodies, soluble receptors, or blocking agents each having a concentration of about 0.1 mg/mL, about 0.2 mg/mL, about 0.5 mg/mL, about 1 mg/mL, about 2 mg/mL, about 5 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 500 mg/mL, or even greater for each of the one or more anti-cytokine antibodies, anti-receptor antibodies, soluble receptors, or blocking agents.

Another embodiment described herein is a pharmaceutical composition comprising one or more anti-cytokine antibodies, anti-receptor antibodies, soluble receptors, or blocking agents (particularly any from Table 1), comprising each protein at the indicated dose. In one aspect, the amount of each protein in the composition is independently increased between 1.5-fold to 50-fold of the indicated dose, including each integer within the specified range. In another aspect, the amount of each protein in the composition is independently increased about 1.5 fold; about 2-fold, about 3-fold, about 4-fold; about 5-fold, about 6-fold; about 7-fold; about 8-fold, about 10-fold, about 20-fold, about 50-fold, or about 100-fold of the indicated dose.

The frequency of dosing will depend upon the pharmacokinetic parameters of the therapeutic agent being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The phrases and terms “can be administered by injection,” “injectable,” or “injectability” refer to a combination of factors such as a certain force applied to a plunger of a syringe containing the anti-cytokine antibodies, anti-receptor antibodies, soluble receptors, or blocking agents described herein dissolved in a liquid at a certain concentration (w/v) and at a certain temperature, a needle of a given inner diameter connected to the outlet of such syringe, and the time required to extrude a certain volume of the anti-cytokine antibodies, anti-receptor antibodies, soluble receptors, or blocking agents from the syringe through the needle.

In one embodiment, an injectability measurement is carried out for the anti-cytokine antibodies, anti-receptor antibodies, soluble receptors, or blocking agents suspended in PBS or physiological saline to a concentration of about 0.1% to about 20% (w/v) including all integers within the specified percentage range.

Another embodiment described herein is a pharmaceutical composition of the anti-cytokine antibodies, anti-receptor antibodies, soluble receptors, or blocking agents described herein. The pharmaceutical compositions can comprise one or more excipients, such as:

(i) Buffering agents: physiologically tolerated buffers to maintain pH in a desired range, such as sodium phosphate, bicarbonate, succinate, histidine, citrate and acetate, sulphate, nitrate, chloride, pyruvate. Antacids such as Mg(OH)₂ or ZnCO₃ may be also used. Buffering capacity may be adjusted to match the conditions most sensitive to pH stability.
(ii) Isotonicity modifiers: to minimize pain that can result from cell damage due to osmotic pressure differences at the injection depot. Glycerin and sodium chloride are examples. Effective concentrations can be determined by osmometry using an assumed osmolality of 285-315 mOsmol/kg for serum.
(iii) Preservatives and/or antimicrobials: multidose parenteral preparations may require the addition of preservatives at a sufficient concentration to minimize the risk of subjects becoming infected upon injection and corresponding regulatory requirements have been established. Typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride.
(iv) Stabilizers: Stabilization is achieved by strengthening of the protein-stabilising forces, by destabilization of the denatured state, or by direct binding of excipients to the protein. Stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives. In addition, oligomers or polymers such as cyclodextrins, dextran, dendrimers, polyethylene glycol, polyvinylpyrrolidone, protamine, or human serum albumin may be used.
(v) Anti-adsorption agents: Mainly ionic or ion-ionic surfactants or other proteins or soluble polymers are used to coat or adsorb competitively to the inner surface of the composition's container, e.g., poloxamer (Pluronic F-68), PEG dodecyl ether (Brij 35), polysorbate 20 and 80, dextran, polyethylene glycol, PEG-polyhistidine, BSA and HSA and gelatines. Chosen concentration and type of excipient depends on the effect to be avoided but typically, a monolayer of surfactant is formed at the interface just above the CMC value.
(vi) Lyophilization or cryoprotectants: During freeze- or spray drying, excipients may counteract the destabilising effects caused by hydrogen bond breaking and water removal. For this purpose, sugars and polyols may be used, but corresponding positive effects have also been observed for surfactants, amino acids, non-aqueous solvents, and other peptides. Trehalose is particularly efficient at reducing moisture-induced aggregation and also improves thermal stability potentially caused by exposure of protein hydrophobic groups to water. Mannitol and sucrose may also be used, either as sole lyo/cryoprotectant or in combination with each other where higher ratios of mannitol or sucrose are known to enhance physical stability of a lyophilized cake. Mannitol may also be combined with trehalose. Trehalose may also be combined with sorbitol or sorbitol may be used as the sole protectant. Starch or starch derivatives may also be used.
(vii) Oxidation protection agents: antioxidants such as ascorbic acid, ectoine, methionine, glutathione, monothioglycerol, morin, polyethylenimine (PEI), propyl gallate, vitamin E, chelating agents such aus citric acid, EDTA, hexaphosphate, thioglycolic acid.
(viii) Viscosifiers or viscosity enhancers: retard settling of the particles in the vial and syringe and are used in order to facilitate mixing and resuspension of the particles and to make the suspension easier to inject (i.e., low force on the syringe plunger). Suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satiagum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly (oxypropylene)-poly(oxyethylene) (e.g., Pluronic.™), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly(acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone. Such block copolymers as well as the abovementioned poloxamers may exhibit reverse thermal gelation behavior (fluid state at room temperature to facilitate administration and gel state above sol-gel transition temperature at body temperature after injection). (ix) Diffusion agents: modifies the permeability of connective tissue through the hydrolysis of components of the extracellular matrix in the interstitial space such as, but not limited to, hyaluronic acid, a polysaccharide found in the intercellular space of connective tissue. A spreading agent such as, but not limited to, hyaluronidase temporarily decreases the viscosity of the extracellular matrix and promotes diffusion of injected drugs.
(x) Other auxiliary agents: such as wetting agents, viscosity modifiers, antibiotics, hyaluronidase. Acids and bases such as hydrochloric acid and sodium hydroxide are auxiliary agents necessary for pH adjustment during manufacture.

In one embodiment, the anti-cytokine antibodies, anti-receptor antibodies, soluble receptors, or blocking agents are sufficiently dosed in the composition to provide therapeutically effective amounts of biologically active agent for at least 12 hours in one application. In one aspect, one application of anti-cytokine antibodies, anti-receptor antibodies, soluble receptors, or blocking agents is sufficient for about 1 day, about 2 days, about 3 days, about 4 days, about 5 days, about 6 days, about 7 days, about 1 week, about 2 weeks, about 3 weeks, about 4 weeks, one month, 2 months, 3 months, 4 months, 6 months, 9 months, one year, 2 years, 3 years, 4 years, or even longer.

In one embodiment, the anti-cytokine antibodies, anti-receptor antibodies, soluble receptors, or blocking agents are provided as a single dose, meaning that the container in which it is supplied contains one pharmaceutical dose.

In another embodiment, the composition is provided as a multiple dose composition, meaning that it contains more than one therapeutic dose. Preferably, a multiple dose composition contains at least 2 doses. Such multiple dose compositions can be used for different subjects in need thereof or for use in one subject, wherein the remaining doses are stored until needed after the administration of the first dose.

In another embodiment, the anti-cytokine antibodies, anti-receptor antibodies, soluble receptors, or blocking agents are provided in one or more containers. For liquid or suspension compositions, the container is preferably a single chamber syringe. For dry compositions, preferably the container is a dual-chamber syringe. The dry composition is provided in a first chamber of the dual-chamber syringe and reconstitution solution is provided in a second chamber of the dual-chamber syringe.

Prior to administering a dry or lyophilized composition of anti-cytokine antibodies, anti-receptor antibodies, soluble receptors, or blocking agents to a subject in need thereof, the dry composition is reconstituted. Reconstitution can take place in the container in which the dry composition is provided, such as in a vial, syringe, dual-chamber syringe, ampoule, and cartridge. Reconstitution is done by adding a predefined amount of reconstitution solution to the dry composition. Reconstitution solutions are sterile liquids, such as phosphate buffered saline, isotonic saline, water for injection, or other buffers, which may contain further excipients, such as preservatives and/or antimicrobials, such as, for example, benzylalcohol and cresol. Preferably, the reconstitution solution is sterile water for injection. Alternatively, the reconstitution solution is physiological saline or sterile phosphate buffered saline (PBS).

Another embodiment is a method of preparing a reconstituted composition comprising a therapeutically effective amount of anti-cytokine antibodies, anti-receptor antibodies, soluble receptors, or blocking agents, and optionally one or more pharmaceutically acceptable excipients, the method comprising the step of contacting the composition with a volume of reconstitution vehicle. The reconstituted anti-cytokine antibodies, anti-receptor antibodies, soluble receptors, or blocking agents may then be administered by injection or other routes.

Another embodiment is a reconstituted composition comprising a therapeutically effective amount of anti-cytokine antibodies, anti-receptor antibodies, soluble receptors, or blocking agents, a reconstitution vehicle, and optionally one or more pharmaceutically acceptable excipients.

Another embodiment is a pre-filled syringe comprising a solution or a suspension comprising a therapeutically effective amount of anti-cytokine antibodies, anti-receptor antibodies, soluble receptors, or blocking agents, and optionally one or more pharmaceutically acceptable excipients. In one aspect, the syringe is filled with between about 0.01 mL and about 50 mL of anti-cytokine antibodies, anti-receptor antibodies, soluble receptors, or blocking agents as described herein. In one aspect, the syringe is filled with between about 0.05 mL and about 50 mL, between about 1 mL and about 20 mL, between about 1 mL and about 10 mL, between about 1 mL and about 5 mL, or about 0.5 to about 5 mL. In one embodiment, the syringe is filled with 0.5 mL to about 2 mL of anti-cytokine antibodies, anti-receptor antibodies, soluble receptors, or blocking agents as described herein. In some aspects, a syringe is filled with about 0.1 mL, about 0.2 mL, about 0.3 mL, about 0.4 mL, 0.5 mL, about 0.6 mL, about 0.7 mL, about 0.8 mL, about 0.9 mL, about 1 mL, about 1.2 mL, about 1.5 mL, about 1.75 mL, about 2 mL, about 3 mL, about 4 mL, about 5 mL, about 7.5 mL, about 10 mL, about 15 mL, about 20 mL, about 25 mL, about 30 mL, about 40 mL, about 50 mL, or greater than 50 mL of anti-cytokine antibodies, anti-receptor antibodies, soluble receptors, or blocking agents as described herein. A syringe is often filled with more than the desired dose to be administered to the patient, to take into account wastage due to “dead space” within the syringe and needle. There may also be a pre-determined amount of waste when the syringe is primed by the physician, so that it is ready to inject the patient.

In one embodiment, a syringe is filled with a dosage volume (i.e., the volume of medicament intended for delivery to the patent) of between about 0.01 mL and about 5 mL depending on the route of injection (e.g., between about 0.01 mL and about 0.1 mL, between about 0.1 mL and about 0.5 mL, between about 0.2 mL and about 2 mL, between about 0.5 mL and about 5 mL, or between about 1 mL and about 5 mL) of about 0.6 mL, about 0.7 mL, about 0.8 mL, about 0.9 mL, about 1 mL, about 1.2 mL, about 1.5 mL, about 1.75 mL, about 2 mL, as described herein. In one embodiment intended for subcutaneous injection, a syringe is filled with a dosage volume of between about 0.1 mL and about 5.0 mL of anti-cytokine antibodies, anti-receptor antibodies, soluble receptors, or blocking agents with concentration(s) of 0.1 mg/mL to 500 mg/mL as described herein. In other embodiments intended for injection by other routes, a syringe is filled with a dosage volume of between about 0.01 mL and about 5.0 mL of anti-cytokine antibodies, anti-receptor antibodies, soluble receptors, or blocking agents with a drug concentration of 0.1 mg/mL to 200 mg/mL as described herein. In some aspects, a syringe is filled with about 0.01 mL, about 0.02 mL, about 0.03 mL, about 0.04 mL, about 0.05 mL, about 0.06 mL, about 0.07 mL, about 0.08 mL, about 0.09 mL, about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.2 mL, about 1.5 mL, about 1.75 mL, about 2 mL, about 2.5 mL, about 3 mL, about 4 mL, or about 5 mL of anti-cytokine antibodies, anti-receptor antibodies, soluble receptors, or blocking agents as described herein for delivery to a patient in need thereof.

As the syringe contains a medicament solution, the outlet may be reversibly sealed to maintain sterility of the medicament. This sealing may be achieved by a sealing device as is known in the art, such as a luer lock or a tamper resistant seal.

Another embodiment is a kit comprising one or more pre-filled syringes comprising a solution or suspension of one or more anti-cytokine antibodies, anti-receptor antibodies, soluble receptors, or blocking agents as described herein. In one embodiment, such a kit comprises a pre-filled syringe comprising anti-cytokine antibodies, anti-receptor antibodies, soluble receptors, or blocking agents as described herein in a blister pack or a sealed sleeve. The blister pack or sleeve may be sterile on the inside. In one aspect, pre-filled syringes as described herein may be placed inside such blister packs or sleeves prior to undergoing sterilization, for example terminal sterilization.

Such a kit may further comprise one or more needles for administration of anti-cytokine antibodies, anti-receptor antibodies, soluble receptors, or blocking agents as described herein. Such kits may further comprise instructions for use, a drug label, contraindications, warnings, or other relevant information. One embodiment described herein is a carton or package comprising one or more pre-filled syringes comprising one or more anti-cytokine antibodies, anti-receptor antibodies, soluble receptors, or blocking agents as described herein contained within a blister pack, a needle, and optionally instructions for administration, a drug label, contraindications, warnings, or other relevant information.

A terminal sterilization process may be used to sterilize the syringe and such a process may use a known process such as an ethylene oxide or a hydrogen peroxide (H₂O₂) sterilization process. Needles to be used with the syringe may be sterilised by the same methods as kits described herein. In one aspect, a package is exposed to the sterilising gas until the outside of the syringe is sterile. Following such a process, the outer surface of the syringe may remain sterile (whilst in its blister pack) for up to 6 months, 9 months, 12 months, 15 months, 18 months, 24 months or longer. Thus, in one embodiment, a pre-filed syringe as described herein (in its blister pack) may have a shelf life of up to 6 months, 9 months, 12 months, 15 months, 18 months, 24 months, or even longer. In one embodiment, less than one syringe in a million has detectable microbial presence on the outside of the syringe after 18 months of storage. In one aspect, the pre-filled syringe has been sterilised using ethylene oxide with a Sterility Assurance Level of at least 10′. In another aspect, the pre-filled syringe has been sterilised using hydrogen peroxide with a Sterility Assurance Level of at least 10′. Significant amounts of the sterilising gas should not enter the variable volume chamber of the syringe. The term “significant amounts” As used herein, refers to an amount of gas that would cause unacceptable modification of the solution or suspension of anti-cytokine antibodies, anti-receptor antibodies, soluble receptors, or blocking agents within the variable volume chamber. In one embodiment, the sterilization process causes <10% (preferably ≤5%, ≤3%, ≤1%) alkylation of the anti-cytokine antibodies, anti-receptor antibodies, soluble receptors, or blocking agents. In one embodiment, the pre-filled syringe has been sterilised using ethylene oxide, but the outer surface of the syringe has <1 ppm, preferably <0.2 ppm ethylene oxide residue. In one embodiment, the pre-filled syringe has been sterilised using hydrogen peroxide, but the outer surface of the syringe has <1 ppm, preferably <0.2 ppm hydrogen peroxide residue. In another embodiment, the pre-filled syringe has been sterilised using ethylene oxide, and the total ethylene oxide residue found on the outside of the syringe and inside of the blister pack is <0.1 mg. In another embodiment, the pre-filled syringe has been sterilised using hydrogen peroxide, and the total hydrogen peroxide residue found on the outside of the syringe and inside of the blister pack is <0.1 mg.

Another aspect is a kit of parts. For liquid and suspension compositions, and when the administration device is simply a hypodermic syringe, the kit may comprise a syringe, a needle, and a container comprising the anti-cytokine antibodies and soluble receptors for use with the syringe. In case of a dry composition, the container may have one chamber containing the dry composition of anti-cytokine antibodies, anti-receptor antibodies, soluble receptors, or blocking agents, and a second chamber comprising a reconstitution solution. In one embodiment, the injection device is a hypodermic syringe adapted so the separate container with the anti-cytokine antibodies and soluble receptors can engage with the injection device such that in use the liquid or suspension or reconstituted dry composition in the container is in fluid connection with the outlet of the injection device. Examples of administration devices include but are not limited to hypodermic syringes and pen injector devices. Particularly preferred injection devices are the pen injectors, in which case the container is a cartridge, preferably a disposable cartridge.

Another embodiment comprises a kit comprising a needle and a container containing the anti-cytokine antibodies, anti-receptor antibodies, soluble receptors, or blocking agents and optionally further containing a reconstitution solution, the container being adapted for use with the needle. In one aspect, the container is a pre-filled syringe. In another aspect, the container is dual chambered syringe.

Another embodiment is a cartridge containing a composition of anti-cytokine antibodies, anti-receptor antibodies, soluble receptors, or blocking agents as described herein for use with a pen injector device. The cartridge may contain a single dose or plurality of doses of anti-cytokine antibodies and soluble receptors.

It will be apparent to one of ordinary skill in the relevant art that suitable modifications and adaptations to the compositions, formulations, methods, processes, and applications described herein can be made without departing from the scope of any embodiments or aspects thereof. The compositions, methods, and experiments provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

### EXAMPLES

### Example 1

All animal studies conducted were approved by the IACUC at Rush University or the University of Alabama at Birmingham. Mouse models were obtained from the following sources: Balb/cJ (Jackson labs #000651), Balb/c (Harlan Labs, Envigo, strain BALB/cAnNHsd), NPHS2-Cre (129S6.Cg-Tg(NPHS2-cre)295Lbh/BroJ; Jackson labs #008523), Enpep −/− mice in mixed 129/C57BL/6 background (kind gift from Drs. Max Cooper and Wadih Arap), C57Bl/6 (Charles River #027). Enpep −/− mice were backcrossed 10 generations into the C57Bl/6 background. Mouse sperm with a floxed Zhx2 construct (zhx2^{tm1a(KOMP)Wtsi}) was obtained from the UC Davis KOMP repository, and foxed mice generated at the UAB Kaul Center for Genetics. Podocyte specific Zhx2 deficient mice were generated by breeding the NPHS2-cre mouse with the foxed mice, genotyped and the protein expression characterized by Western blot of Dynabead™ isolated glomeruli and confocal imaging of kidney sections.

Recombinant mouse cytokines or soluble receptors were purchased: Interferon-γ (Millipore Sigma # IF005); Tumor Necrosis Factor-α (Sigma-Aldrich # T7539); Interleukin-10 (Sigma Aldrich #13019); IL-6 (Sigma Aldrich # SRP3330); ICAM-1 (R&D Systems #796-IC-050); IL-4R (R&D Systems #530-MR-100); IL-2 (R&D Systems #402-ML-020/CF).

A cytokine cocktail was produced by combining the following cytokines and soluble receptors (IL-2, IL-4R, IL-6, IL-10, INF-γ, TNFα, ICAM-1) in a final volume of 100 μL of sterile 0.9% saline (concentration/dose: 1× or X): 0.5 μg of IL-10; 1 μg each of IL-6 and TNFα; and 2 μg each of IL-2, IL-4R, INF-γ, and ICAM-1. Progressively lower doses of the cytokine cocktail were prepared by serially diluting the cytokine cocktail in 100 μL of sterile 0.9% saline as follows: X/2 (2-fold dilution), X/5 (5-fold dilution), X/10 (10-fold dilution), X/15 (15-fold dilution), and X/100 (100-fold dilution).

Mice were injected with 100 μL of the cocktail or dilution intravenously. An additional dose of 100 μL of 0.9% saline was administered intraperitoneally immediately after the intravenous cytokine cocktail dose to maintain intravascular volume.

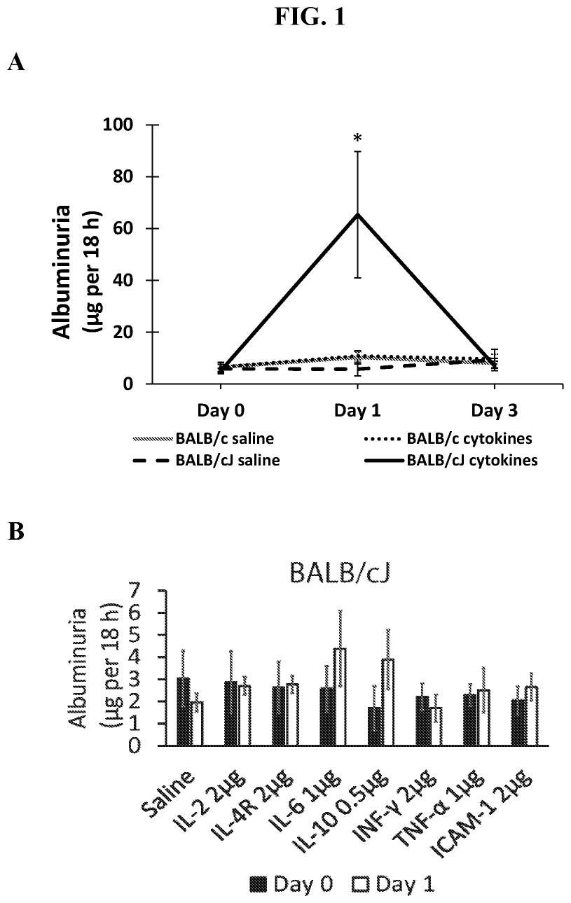

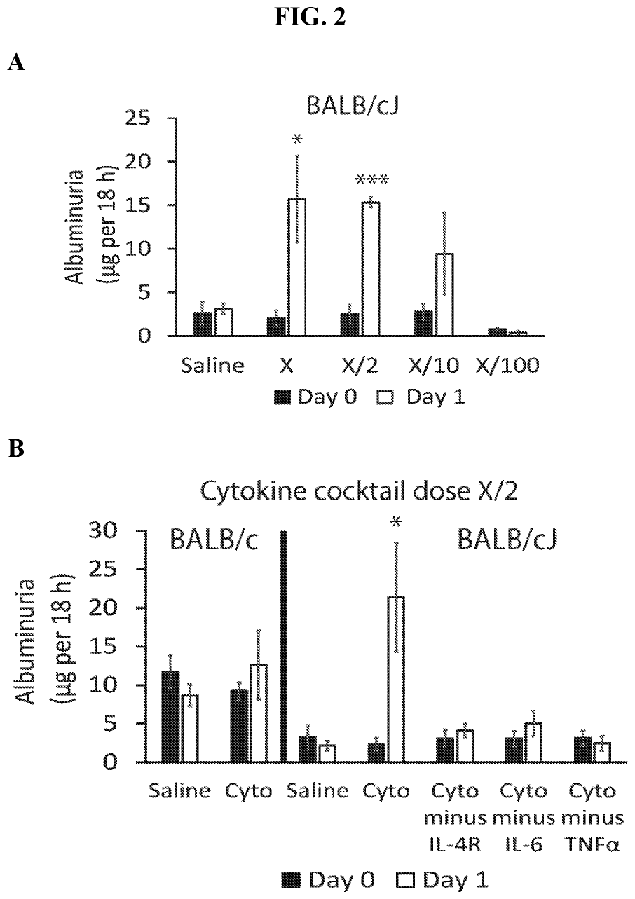

The cytokine cocktail induces acute albuminuria in Balb/cJ, but not in Balb/c mice, at combination dose X (FIG. 1A). Balb/cJ mice have reduced expression of ZHX2 and its transmembrane partners. None of the individual cytokines administered at the same dosage as in the cocktail at 1× concentration caused albuminuria in either mouse strain (FIG. 1B). In order to establish a minimum nephritogenic dose, fractions of dose X were injected and the mice assessed for albuminuria. Reducing the combination dose to 50% or 20% of X still induced significant albuminuria in Balb/cJ mice (FIG. 2A). At 10% of combination X dose, albuminuria was present but not statistically significant for the small number of animals tested. At 1% of combination X dose, no albuminuria was observed in Balb/cJ mice (FIG. 2B).

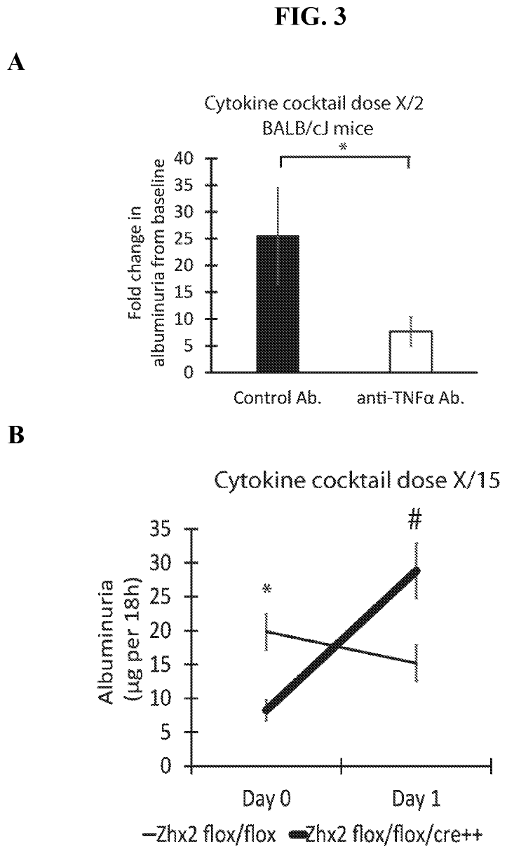

The elimination of one of the three specific cytokines or soluble receptors against which antibodies are in clinical use (IL-6, TNFα, IL-4R) individually from the cocktail of cytokines failed to induce albuminuria in Balb/cJ mice (FIG. 3A). This indicated that neutralization of any of these cytokines could inhibit acute relapse of glomerular disease. The injection of Balb/cJ mice with an anti-rat TNFα antibody (25 μg) or a control antibody (IgG) one hour after injection of cytokine cocktail dose (X/2) reduced the intensity of cytokine cocktail induced albuminuria in Balb/cJ mice in the anti-TNFα group as compared to the IgG control (FIG. 3B). Using a larger dose of anti-TNFα antibody could eliminate cytokine cocktail induced albuminuria.

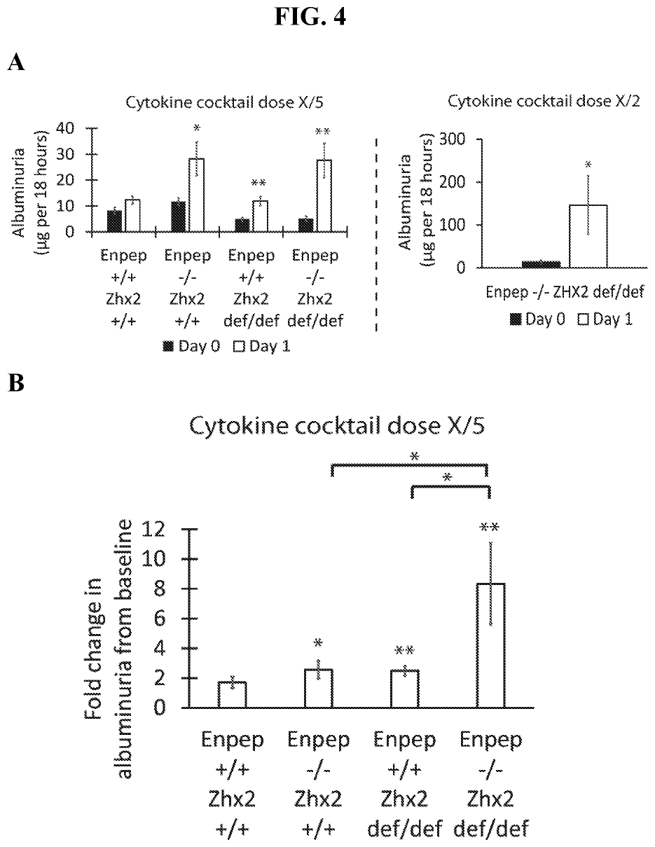

Using the minimum nephritogenic dose model, cytokine induced albuminuria was also noted in ZHX2 flox/flox cre⁺⁺, but not control ZHX2 flox/flox mice (FIG. 4A). This finding indicates the specific role of podocyte ZHX2 deficiency in this process. Mice deficient in Enpep also had cytokine induced albuminuria, whereas mice deficient in both ZHX2 and Enpep had the highest fold-increase in albuminuria (FIG. 4B). Using higher doses of cytokines increased albuminuria further (FIG. 4A, right panel).

The rat cytokine cocktail contained recombinant rat cytokines or soluble receptors purchased from the following sources: Interferon-γ (R&D #585-IF/CF); Tumor Necrosis Factor-α (Sigma-Aldrich # T5944-50μ); Interleukin-10 (R&D #522-RLB); IL-6 (R&D #506-RL/CF); ICAM-1 (R&D Systems #583-IC); IL-4R (Sino Biological #80198-R08H); IL-2 (Sigma Aldrich # SRP3242-20). A 1× dose of the cocktail included 0.5 μg of IL-10, 1 μg each of IL-6 and ICAM-1, and 2 μg each of IL-2, IL-4R, INF-γ and TNFα dissolved in a final volume of 100 μL of sterile 0.9% saline. The minimum nephritogenic dose at X/50 contained the appropriate fraction of each cytokine in 100 μL of sterile 0.9% saline. During Buffalo Mna rat cytokine studies, male rats (n=7) with baseline proteinuria between 35-63 mg/18 hours were injected intravenously with X/50 cytokine cocktail dose, followed immediately by 1 mL of 0.9% saline intraperitoneally to maintain intravascular hydration. Timed urine collections (18 hours) were conducted on days 1, 3, 5 and 7, and the peak increase in proteinuria noted for each animal.

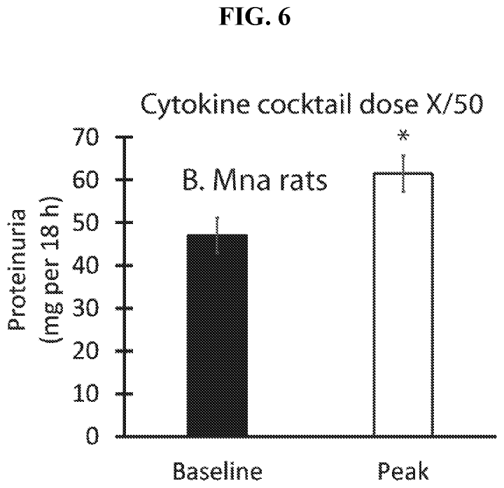

Despite being about 20-fold heavier than mice, Buffalo/Mna rats with active focal and segmental glomerulosclerosis (FSGS) needed a much lower dose (X/50) of the rat “cytokine cocktail” to induce a significant increase in proteinuria from baseline (FIG. 6), mimicking thereby disease worsening following a common cold.

### Example 2

The zinc-finger and homeobox (ZHX) family transcriptional factors (ZHX1, ZHX2, and ZHX3) regulate a majority of the structurally and functionally important genes expressed in kidney podocytes. The majority of ZHX proteins in in vivo podocyte are located outside the nucleus and tethered to the cell membrane as hetero- or homo-dimers (e.g., ZHX1-ZHX2 or ZHX1-ZHX1). Loss of ZHX hetero- or homo-dimerization results in the entry of ZHX proteins into the nucleus, followed by changes in the expression of target genes.

Zhx2 is the major transcriptional repressor of α-fetoprotein expression in adult mice. Balb/cJ mice, but not 25 other mouse strains studied, have a mouse endogenous retrovirus in the first intron of the Zhx2 gene that results in a predominantly non-functional transcript. This leads to Zhx2 downregulation and high α-fetoprotein protein levels in adult Balb/cJ mice.

Confocal imaging of mouse glomeruli was performed using a Zeiss Pascal 5 confocal laser microscope. All secondary antibodies were purchased from Jackson labs. Published primary antibodies include rabbit anti-ZHX3 (4457-2B5). The following primary antibodies were purchased: mouse anti-WT1 (Santa Cruz # sc-7385); mouse anti-ZHX2 (Abnova, Taiwan # H00022882-A01); and rabbit anti-ZHX3 (Santa Cruz # sc-292339).

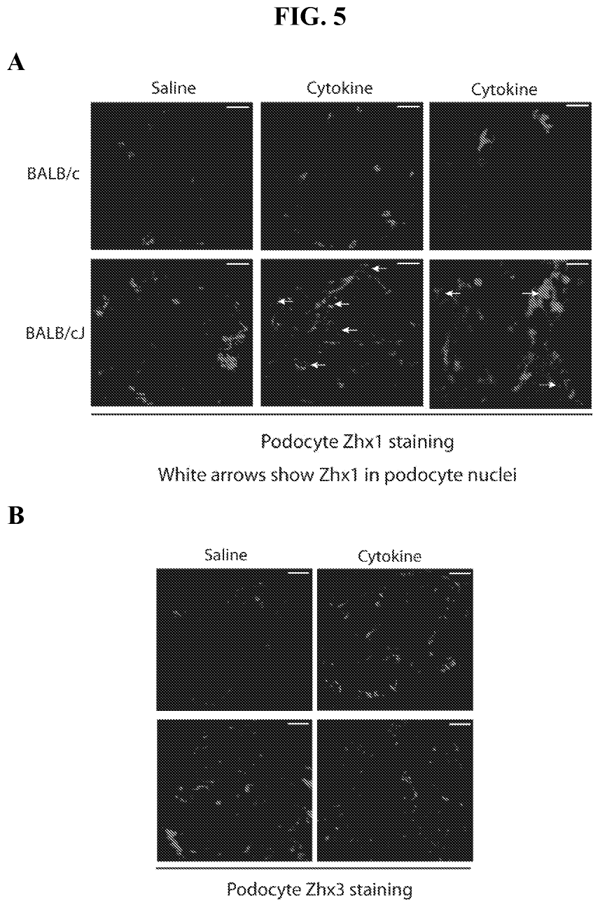

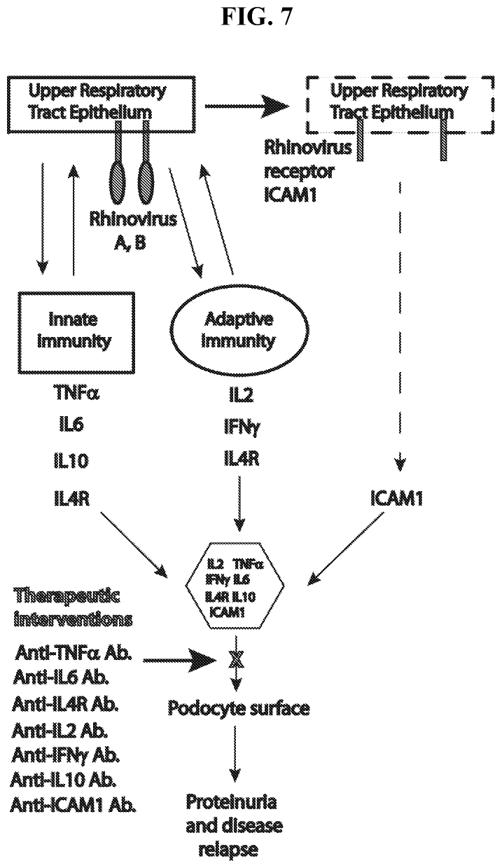

Balb/cJ mice injected with cytokines had higher nuclear expression of Zhx1 (FIG. 5A,), whereas nuclear Zhx3 expression was not increased (FIG. 5B), suggesting thereby that cytokine mediated albuminuria was largely mediated through the podocyte body surface pathways as would happen in minimal change disease. A summary of this paradigm is shown in FIG. 7.

A cocktail of cytokines and soluble receptors present in circulation during a common cold was developed to study the frequently observed phenomenon of relapse or worsening of primary glomerular diseases after a common cold. The original dose combination developed could be diluted several fold and retain the ability to induce albuminuria in podocyte Zhx2 deficient, Enpep deficient or dual deficient mice. Individual cytokines did not induce albuminuria, nor did the individual elimination of three cytokines or soluble receptors (IL-2, IL-4R, IL-6, IL-10, INF-γ, TNFα, ICAM-1) from the cocktail with commercially available antibodies. These results encourage future clinical studies on early treatment of individuals susceptible to common cold induced relapse with commercially available antibodies against IL-2, IL-4R, IL-6, IL-10, INF-γ, TNFα, or ICAM-1 to stem relapse.

What is claimed:

1. A method for inhibiting acute relapse or worsening of a glomerular disease initiated by a viral infection, the method comprising administration to a subject in need thereof of one or more anti-cytokine antibodies, anti-receptor antibodies, soluble receptors, or blocking agents, wherein the subject is in complete or partial remission of the glomerular disease prior to the viral infection and the viral infection comprises a virus that induce a common cold or flu.

2. The method of claim 1, wherein the one or more anti-cytokine antibodies, anti-receptor antibodies, soluble receptors, or blocking agents comprise one or more of anti-IL-2, anti-IL-6, anti-IL-10, anti-INF-γ, anti-TNFα, soluble TNF receptors, anti-IL-4R, anti-IL-4Ra, anti-IL-6 receptor, anti-ICAM-1, or combinations thereof.

3. The method of claim 1, wherein the one or more anti-cytokine antibodies, anti-receptor antibodies, soluble receptors, or blocking agents comprise one or more of anti-IL-4R, anti-IL-6, anti-IL-6 receptor, anti-TNFα, soluble TNF receptors, or combinations thereof.

4. The method of claim 1, wherein the one or more anti-cytokine antibodies, anti-receptor antibodies, soluble receptors, or blocking agents comprise one or more of infliximab (REMICADE®), adalimumab (HUMIRA®), certolizumab pegol (CIMZIA®), etanercept (ENBREL®), siltuximab (SYLVANT®), tocilizumab (ACTEMRA®), dupilumab (DUPIXENT®), afelimomab, bersanlimab, blazakizumab, fontolizumab, golimumab, nerelimomab, olokizumab, ozoralizumab, pascolizumab, placulumab, sapelizumab, sarilumab, vobarilizumab, combinations thereof, derivatives thereof, or analogues thereof.

5. The method of claim 1, wherein the glomerular disease comprises nephrotic syndrome.

6. The method of claim 1, wherein the glomerular disease comprises minimal change disease, focal segmented glomerulosclerosis, membranous nephropathy, membranoproliferative glomerulonephritis, diabetic nephropathy, or lupus nephritis.

7. The method of claim 1, wherein the glomerular disease comprises acute relapse or worsening of chronic kidney disease caused by minimal change disease, focal segmented glomerulosclerosis, membranous nephropathy, membranoproliferative glomerulonephritis, diabetic nephropathy, or lupus nephritis.

8-9. (canceled)

10. The method of claim 1, wherein viral infection comprises rhinovirus, human coronavirus, adenovirus, respiratory syncytial virus, enteroviruses other than rhinoviruses, parainfluenza virus, metapneumovirus, influenza, or combination thereof.

11. (canceled)

12. The method of claim 1, wherein administration occurs no later than about₇ days after of onset of the viral infection.

13. The method of claim 1, wherein the administration comprises parenteral administration.

14. The method of claim 1, wherein the administration comprises intravenous, intraarterial, intramuscular, intradermal, subcutaneous, or intraperitoneal administration.

15. (canceled)

16. The method of claim 1, wherein the subject is a human.

17. A method for depleting a subject's systemic levels of one or more of a cytokine selected from the group consisting of IL-2, IL-6, IL-10, INF-γ, or TNFα, or inhibiting a receptor for IL-4R or ICAM-1, the method comprising contacting the cytokines or receptors with one or more anti-cytokine antibodies, anti-receptor antibodies, soluble receptors, or blocking agents, wherein the subject is in complete or partial remission of glomerular disease prior to experiencing the acute relapse onset or worsening of glomerular disease.

18. The method of claim 17, wherein the one or more anti-cytokine antibodies, anti-receptor antibodies, soluble receptors, or blocking agents comprise anti-IL-2, anti-IL-6, anti-IL-10, anti-INF-γ, anti-TNFα, anti-IL-4R, anti-ICAM-1, or combinations thereof.

19. The method of claim 17, wherein the contacting comprises parenteral administration of the one or more of anti-cytokine antibodies, anti-receptor antibodies, soluble receptors, or blocking agents.

20. The method of claim 19, wherein the parenteral administration comprises intravenous, intraarterial, intramuscular, intradermal, subcutaneous, or intraperitoneal administration.

21-23. (canceled)

24. The method of claim 17, wherein the subject is a human.

25-32. (canceled)

33. A kit for depleting a subject's systemic levels of one or more of cytokines selected from the group consisting of IL-2, IL-6, IL-10, INF-γ, or TNFα or inhibiting a IL-4R, or ICAM-1 the kit comprising one or more receptacles comprising a mixture of one or more anti-cytokine antibodies, anti-receptor antibodies, soluble receptors, or blocking agents comprising anti-IL-2, anti-IL-6, anti-IL-10, anti-INF-γ, anti-TNF, anti-IL-4R, anti-ICAM-1, blocking agents, or a combination thereof, and optionally, a delivery device, a diluent, instructions for use, or a label.
